(19) United States
(12) Patent Application Publication
(10) Pub. No.: US 2020/0143928 A1
(43) Pub. Date: May 7, 2020

(54) Pictogram Medication Code and Education Tool to Improve Patient Adherence

(71) Applicant: Amy Cypora Rothkopf, West Caldwell, NJ (US)

(72) Inventor: Amy Cypora Rothkopf, West Caldwell, NJ (US)

(21) Appl. No.: 16/675,110

(22) Filed: Nov. 5, 2019

Related U.S. Application Data
(60) Provisional application No. 62/756,920, filed on Nov. 7, 2018.

(51) Int. Cl. G16H 20/17; G16H 10/60; G16H 70/40

(52) U.S. Cl. CPC G16H 20/17; G16H 70/40; G16H 10/60

(57) ABSTRACT

A system for educating or assisting patients or caregivers in administering the proper medication and dosages of medication to a patient comprising a simple pictogram assigned to a medication, a master medication list comprising an identifier of the pictogram, an identifier of the medication, and an indicator that the pictogram is assigned to the medication, and a container of the medication labeled with the assigned pictogram.

### RELATED APPLICATIONS

This application is a nonprovisional patent application claiming priority to pending U.S. Provisional Patent Application No. 62/756,920, filed by me on Nov. 7, 2018. This entire disclosure of U.S. Provisional Patent Application No. 62/756,920 and the drawings submitted therewith are incorporated herein by reference.

### SUMMARY

This medication toolkit assigns a pictogram code to each medication, detailed in a patient-friendly medication list. Each medication bottle is labelled at the pharmacy with a matching pictogram corresponding to the medication list. This allows patients with low literacy to easily identify and refer to medications. Coded pill organizers and oral syringes are also available to help simplify administration. Electronic medical record (EMR) integration enables all pharmacists and providers, even in their first encounter with a patient, to immediately reconcile the current medication regimen with the patient using the code. It also ensures that providers, pharmacists, and patients all have access to the same code to facilitate accurate and timely communication. The system described herein further facilitates patient and caregiver education and communication, increasing the likelihood of a patient's adherence to his or her medication regimen, which is associated with improved health outcomes.

### BACKGROUND

Studies show that patients who adhere to medication regimens experience better health outcomes. Adherence is a simple concept but is an often-overlooked component of high quality health care and can be challenging for patients with low literacy skills, inadequate health education, and/or low English proficiency skills. Furthermore, in chronic disease health states, medication regimens are constantly changing, especially when new therapies are added, and at points of transition of care.

This tool was designed in the context of preventing rejection in organ transplant recipients, which significantly limits long-term survival, impairs quality of life, and increases both direct and indirect costs of care. The only way to prevent rejection is strict adherence to a complex outpatient regimen commonly consisting of 10-12 medications. However, in the United States, many patients have limited literacy or English proficiency, and are unable to follow this medication schedule accurately. A solution was needed to give providers and patient educators a standardized way to teach these complex concepts and improve adherence in low literacy families. As the treatment of many diseases requires complex outpatient pharmaceutical therapy that must be self-directed by the patient and/or caregiver, this tool can be adapted to cater to the management of other chronic diseases, such as diabetes, heart disease, and various cancer treatments, among others.

Typically, providers spend hours teaching patients/caregivers about discharge medications. Non-native English speakers are taught via interpreter in their native language, which, on average, doubles the teaching time required to meet proficiency. Patient education involves repetitive in-depth discussions and demonstrations of how medications should be administered, as well as the indication, dosage, timing, and common side effects.

Patients are routinely provided with a patient-friendly medication list in English, regardless of native language, and ideally patients must demonstrate understanding prior to discharge. There is no way for patients with inadequate English literacy to refer to medicines specifically, other than pointing to the medication list in the presence of the provider. The bulk of outpatient medication adjustments take place via phone, in reaction to laboratory findings, such as therapeutic drug levels. There is no simple way to communicate the complexities of outpatient medication teaching to patients of low English literacy, effectively removing an element of patient/caregiver input in medical decision-making. Patients receive medication refills from a preferred pharmacy, but many times the instructions on the bottle are inaccurate or incomprehensive to patients regardless of their literacy level.

This is the first description of a standardized medication code used by patients and providers to improve communication and compliance. The innovation not only improves patient education and medication adherence, it creates an accessible language for patients and providers to discuss medications, regardless of language, health literacy, or English proficiency. This empowers patients and caregivers to share their experience and participate in medical decision-making in a way that was never before possible. It also simplifies health encounters that are facilitated by phone interpreters, saving time for providers and improving patient satisfaction. Patients and families who speak conversational English, but may not be confident with reading and writing, or have low health literacy can use the code provided to become an active member in the care team, adding a vital perspective in medical decision making. Providers will also benefit, utilizing EMR integration to communicate with patients more efficiently, potentially reducing the dependence on language interpreters.

### BRIEF DESCRIPTION OF THE DRAWINGS

FIG. 1 is a master medication list assigning pictograms to each medication in a list of medications;

FIG. 2 is a medication labeled with a pictogram; and

FIG. 3 is a syringe labeled with a pictogram.

### DESCRIPTION

The present invention addresses the need for clear instructions regarding the administration of outpatient medications for patients with limited literacy or language skills. These patients often cannot distinguish one medicine from another. This issue is particularly problematic where patients have multiple prescription medications. The various prescription medications are often delivered to the patient all at once in identical bottles with near identical labels, making them nearly impossible to distinguish without the ability to read the labels. Furthermore, standard pharmacy labels can be difficult for even literate patients to comprehend, often containing only the generic or brand name, and in small font size.

A patient or caregiver might memorize the look of a medication to differentiate the medications—for example, a patient might become familiar with the color, size, and shape of a pill—but this alternative becomes unhelpful when medications looks similar or the appearance of a medication changes. In fact, these changes in appearance are not uncommon during the course of treatment. The look of a medication might change, for example, when the insurer or pharmacy chooses to change pharmaceutical manufacturers, change from brand to generic or vice versa, or when a medical provider alters dosage. One manufacturer might color a medication pink, while another colors it blue. Many pharmaceutical manufacturers will color code their medications so that pills for different doses of the same medication are a different color, complicating this even further. These changes in appearance are likely to confuse a patient or caregiver who relies on the look of a medication to identify it.

However, distinguishing and identifying medications is of vital importance since each medication will likely have its own instructions for administration. For example, medications may require that a patient take them at different times of day (including, for example, morning, night, with meals, or at a certain time interval after eating). Medications might also require different doses. One medication dose might be one pill, while another might require two, for example. Due to the variations in administration amongst medications, it is of the utmost importance that a patient or caregiver is able to distinguish between multiple medications.

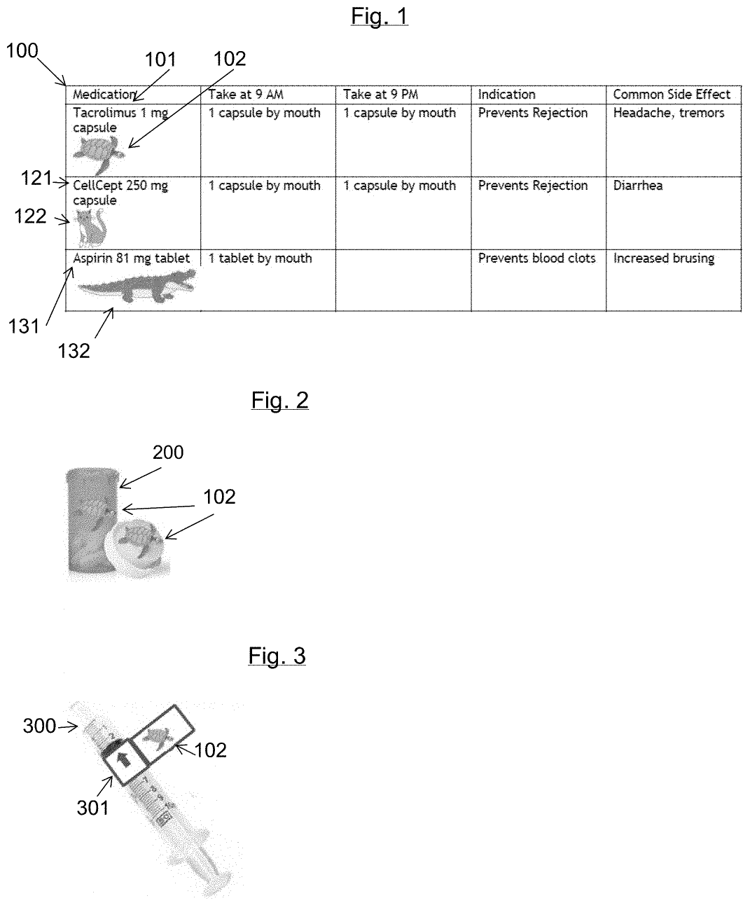

The present invention solves this problem by assigning a simple pictogram 102, 122, 132 to each medication. If a patient has a medicine regimen consisting of tacrolimus, CellCept®, and aspirin, for example, each of these medications will be assigned a different pictogram. This example is illustrated in FIG. 1. As seen in the figure, tacrolimus 101 is assigned a turtle pictogram 102, CellCept® 121 is assigned a cat pictogram 122, and aspirin 131 is assigned an alligator pictogram 132. In the preferred embodiment, pictogram assignments may be based on any number of features of the medication that the patient can easily associate with the medication to help the patient remember the pictograms assignments. This may be matching the first letter of the generic or brand name of the drug to pictogram, as is the example with CellCept® 121 and cat 122. Alternatively, the pictogram may represent any number of characteristics of the medication. This includes but is not limited to: the indication of the medicine, a common side effect, a commonly identified taste of the medicine, a key element in the molecular compound, or even a patient's nickname of the medicine, especially if the actual term is hard to pronounce. Pictograms can be catered to each patient's preferences, and ideally each patient would have their own unique code.

The pictograms 102, 122, 132 are images of simple objects which can be translated and pronounced easily in a number of languages. These objects may be animals, numbers, letters, shapes and so on. There is the possibility to use license images as well for pictograms to make this tool even more user friendly (i.e. licensed Disney® characters as pictograms to cater to pediatric patients). The simplicity and universal characteristics of the pictogram images ensure that a person who speaks any language, or, who simply, such as in the case of small children, cannot read the language that the medicine label is written in, will be able to recognize and refer to the medications as necessary. This is particularly helpful in the cases of immigrant populations and medical tourism, but also helps the general population which is overall unfamiliar with the names and pronunciation of prescription medications. With each pictogram corresponding to a different medication, a patient or caregiver will need to only know the word for the corresponding image to recognize the medication.

The master medication list 100 can be made available to the patient in paper format and/or electronically via a secure smartphone application or patient portal website. Changes that a provider makes to the medication regimen are mirrored in real time in the Electronic medical record (EMR), which is available to other providers and pharmacists, and in the electronic iterations for patient reference. Ideally a new paper copy would be printed for the patient at this time as well, which may be included in the EMR's patient summary document.

The simplicity and ability to pronounce the images also ensures that small children who may be taking the medicines will also be able to recognize the images and therefore the medications, fostering early interest and engagement while still under the supervision of an adult. In this way, the system described herein may be used as educational tool to teach children, or adults for that matter, how to approach medications in general and how to correctly use their medications in particular. In this use, a medical professional can review the medication list with a patient or caregiver, in the office or hospital. The medical professional can explain to the user how to use each medication and ensure that the user understands the regimen before her or she receives the medications.

In the invention, the pictogram is printed on or adhered to the bottle or container 200 comprising the medication such that the medicine is labeled with the pictogram 102, as seen in FIG. 2. In a preferred embodiment, each medication bottle 200 is labelled at the pharmacy, where the printer that creates labels for prescription bottles would be able to print the identical pictogram in color, size, and clarity as it is visualized in the EMR or on paper. Coded pill organizers and syringes 300 are also available to help simplify administration. The medicines 101, 121, 131 and their pictogram 102, 122, 132 are also recorded on a master medication list 100, preferably in a table format, illustrated in FIG. 1 which provides an identifier for medication 101, 121, 131, an identifier for its corresponding pictogram 102, 122, 132, and an indication of which medication corresponds to which pictogram, which, in the example provided in FIG. 1, is grouping the name of the medication and the image of the pictogram together in the mater list 100. The identifier for the medication 101, 121, 131 most commonly is the medication name although it may also include type (anticoagulant for aspirin, for example), tradename (such as CellCept®), generic name, or commonly used abbreviation (such as NaCl for saline). The identifier for the pictogram 102, 122, 132 might be for example an image of the pictogram or the word for the pictogram (turtle, cat, alligator) or another means to identify the pictogram. The master medication list 100 can also include the instructions for administration or other information such as the purpose of each medication or prescribing medical professional. The master medication list may be in multiple languages, include translations, or have multiple versions where each version is in a different language. The master medication list may also contain photos or drawings of the dosage to cater to patients/caregivers who have limited numeracy skills. In a preferred embodiment, the patient's medical teams and pharmacists have a copy of the master medication list so that all medical staff can communicate with the patient, caregiver, or other medical professionals regarding the patient's regimen. In the preferred embodiment, an identifier for the pictograms and an indication of which medicines they represent—or, alternatively, the master list itself—are also integrated into the patient's EMR profile. EMR-integration enables all providers, even in their first encounter with a patient, to immediately reconcile coded medications. It also ensures that providers, pharmacists, and patients, all have access to the same code to facilitate accurate and timely communication. In a preferred embodiment, the master medication list would be available on a web platform or app that is adjacent to the EMR so that changes in medication regimen could be made in real time.

In the case of liquid medications, the same system can be employed to indicate the correct dose of each medication, as illustrated in FIG. 3. A pictogram 102 corresponding to the medication 101 on the master medication list 100 may be placed on the syringe 300, or other tool for administration, to indicate that that syringe goes with the medication of the same pictogram. That pictogram 102 may alternatively or additionally also be placed on the syringe 300 at the point at which the syringe will hold the correct dose. For example, if a patient needs 5 ml of a medicine, the pictogram 102 will be placed at the 5 ml marker on the syringe 300. In an alternative embodiment, the syringe or tool for administration may include more than one pictogram, where the first pictogram 102 identifies the medication according the master medication list 100, and the second pictogram or dosage indicator 301 indicates the proper dose. In this embodiment the second pictogram 301 might be, for example, an arrow, a bright colored line, or a stop sign, which communicates to a user where to fill the syringe 300 for the correct dose.

While this invention has been particularly shown and described with references to preferred embodiments thereof, it will be understood by those skilled in the art that various changes in form and details may be made therein without departing from the spirit and scope of the invention as defined by the following claims.

1. A system for educating or assisting patients or caregivers in administering the proper medication and dosages of medication to a patient comprising:
a simple pictogram assigned to a medication;
a master medication list comprising an identifier of the pictogram, an identifier of the medication, and an indicator that the pictogram is assigned to the medication; and
a container of the medication labeled with the assigned pictogram.

2. The system of claim 1 further comprising instructions for administration of the medication.

3. The system of claim 1, wherein an identifier for the pictogram and an indicator that the pictogram is assigned to the medication are integrated into the patient's EMR profile.

3. The system of claim 3, wherein an identifier for the pictogram and an indicator that the pictogram is assigned to the medication are also integrated into a web application adjacent to the patient's EMR profile.

4. The system of claim 1, wherein the medication is a liquid and further comprising:
a syringe to administer the medication wherein the syringe is labeled with the pictogram assigned to the medication.

5. The system of claim 4 wherein the syringe is labeled with the pictogram at the point corresponding to the proper dosage of the medication.

6. The system of claim 4, wherein the syringe is further labeled with a dosage indicator at the point corresponding to the proper dosage of the medication.

7. A method for educating or assisting patients or caregivers in administering the proper medication and dosages of medication to a patient, comprising:
assigning a simple pictogram to a medication;
providing a master medication list comprising an identifier of the pictogram, an identifier of the medication, and an indicator that the pictogram is assigned to the medication; and
providing a container for the medication labeled with the assigned pictogram.

8. The method of claim 7 further comprising providing instructions for administration of the medication.

9. The method of claim 7 further comprising integrating into the patient's EMR profile an identifier for the pictogram and an indicator that the pictogram is assigned to the medication.

10. The method of claim 9 further comprising integrating into a web application adjacent to the patient's EMR profile an identifier for the pictogram and an indicator that the pictogram is assigned to the medication.

11. The method of claim 7, wherein the medication is a liquid and further comprising:
providing a syringe to administer the medication wherein the syringe is labeled with the pictogram assigned to the medication.

12. The method of claim 11 wherein the syringe is labeled with the pictogram at the point corresponding to the proper dosage of the medication.

13. The method of claim 11, wherein the syringe is further labeled with a dosage indicator at the point corresponding to the proper dosage of the medication.
